(19) United States
(12) Patent Application Publication
(10) Pub. No.: US 2010/0105020 A1
(43) Pub. Date: Apr. 29, 2010

(54) QUANTITATIVE MEASUREMENT OF GLYCATED HEMOGLOBIN

(75) Inventors: Kristiane Anne Schmidt, Eindhoven (NL); Gerhardus Wilhelmus Lucassen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/444,435

(22) PCT Filed: Oct. 23, 2007
(86) PCT No.: PCT/IB2007/054300

§ 371 Date: Apr. 6, 2009

(30) Foreign Application Priority Data
Oct. 24, 2006 (EP) 06301086.2

(51) Int. Cl. C12Q 1/02; C12M 1/34

(52) U.S. Cl. 435/2; 435/288.7

(57) ABSTRACT

The invention relates to a method for quantitative measurement of glycated hemoglobin (HbAIc) in a blood sample containing hemoglobin molecules. In this method, the hemoglobin molecules are extracted from the blood sample, adsorbed onto a roughened metal surface and subjected to a Surface Enhanced Raman Scattering (SERS) measurement. The invention provides a universal method for the measurement of the HbAIc concentration in blood.

### FIELD OF THE INVENTION

The invention relates to the quantitative measurement of glycated hemoglobin.

### BACKGROUND OF THE INVENTION

People suffering from diabetes mellitus need the glucose concentration in their blood to be monitored throughout their life. However, direct glucose detection is not a very reliable index for long-term control, since it is a short-term marker, subjected to very fast fluctuations, due for instance to exercise or recent food ingestion.

Hemoglobin is the protein that transports oxygen through blood, within the red blood cells. It is composed of four protein chains, two alpha-chains and two beta-chains, each with a ring-like heme group containing an iron atom. Oxygen, reversibly bound to the iron atom, is transported through blood.

Most of the hemoglobin—about 97%—is of a type called HbA, which will be designated as “normal” hemoglobin. HbA may be modified into HbA1c, which is formed when glucose molecules and HbA molecules react in a process known as glycosylation. Glycosylation is a non-enzymatic irreversible reaction, which is also sometimes referred to as glycation. HbA1c is therefore called “glycosylated hemoglobin”, “glycated hemoglobin” or “glycohemoglobin”.

It has been demonstrated that, the higher the blood glucose level, the more glycosylation of hemoglobin will occur, while the longer the time during which the blood glucose level is high, the more glycosylation will occur. Therefore, HbA1c levels depend primarily upon time-averaged blood glucose levels and provide a reflection of the glycemic control, which is especially interesting for people with diabetes.

Hemoglobin molecules persist in the blood stream until apoptosis occurs; the HbA1c concentration therefore represents the glucose integrated values over the last six to eight weeks. Hence, the HbA1c concentration is not subject to the very fast fluctuations which can be observed when blood glucose concentrations are directly measured; it is therefore considered as a valuable index in the monitoring of long-term glucose control for diabetic people. The amount of HbA1c is usually expressed as a percentage of total hemoglobin.

There are a number of methods by which HbA1c can be measured, which are based on separating the HbA1c molecules from the HbA molecules. The separation can be based on charge differences—ion-exchange chromatography, high-performance liquid chromatography (based on cation-exchange chromatography), electrophoresis or isoelectric focusing—or structural differences (affinity chromatography or immunoassay), or chemical analysis (photometry or spectrophotometry).

However, such methods cannot be standardized over different laboratories and reference methods as well as reference materials are still under development. As a consequence, measurements made by different laboratories are difficult to compare. Furthermore, in order to be able to interpret the measurements made by a particular laboratory, one needs to know the reference range provided by this laboratory. Besides, the concentration of HbA1c is difficult to measure, since it is usually very low.

Reference WO 2005/064314 discloses the use of Raman scattering for HbA1c detection, but a need exists for improving such a method, since Raman scattering is not very sensitive to discriminate HbA and HbA1c properly. Typically, Raman scattering allows the detection of analytes in the millimolar concentration range, while the HbA1c concentration in the blood of healthy people is in the range of only 0.3 to 0.6 mM.

### SUMMARY OF THE INVENTION

It is therefore an object of the present invention to provide a universal method for quantitative measurement of glycated hemoglobin in a blood sample that permits to obtain reliable measurements.

Accordingly, the present invention relates to a method for quantitative measurement of glycated hemoglobin (HbA1c) in a blood sample containing hemoglobin molecules, the method comprising:
extracting the hemoglobin molecules from the blood sample,
adsorbing the hemoglobin molecules onto a roughened metal surface and
analyzing the spectrum obtained by a Surface Enhanced Raman Scattering of the adsorbed molecules.

An advantage of the invention is that the measurement is reliable. Indeed, differentiation of glycated hemoglobin from normal hemoglobin with Surface Enhanced Raman Scattering is based on changes in the vibrational spectrum due to the different chemical structure of the molecules. Most of the prior art methods relate to charge differences or binding properties which makes them sensible to other molecules with similar properties. Besides, thanks to the surface enhancement, the signals, obtained with the method of the present invention, are sufficient for a reliable measurement.

Another advantage of the invention is that it permits fast analysis of a blood sample. Indeed, since the method of the invention does not require the separation of normal hemoglobin from glycated hemoglobin, which is a time-consuming operation, the measurement may be carried out quite quickly. As a consequence, a patient, visiting a doctor for his blood to be sampled and measured, may have a feedback from the doctor during this same visit, while the prior art methods usually made it necessary to visit the doctor twice.

Another advantage of the present invention is that Surface Enhanced Raman Scattering detects any glucose molecule bound to hemoglobin, not only those bound at special positions of the hemoglobin molecule: the measurement is therefore more accurate.

According to another embodiment of the present invention, Raman scattering is further enhanced by means of Resonance Raman Scattering. The obtained signals are therefore further enhanced.

In this embodiment, if the Raman scattering measurement is performed with a laser, the laser wavelength may be chosen in a normal hemoglobin (HbA) absorption band, e.g. between 400 and 600 nm.

According to a particular embodiment of the present invention, the concentrations of HbA and of HbA1c are determined simultaneously. This is possible since the spectrum yields information on both of them. It is also possible to detect hemoglobin abnormalities, which can be indicated by other variations in the hemoglobin spectrum. Both factors may influence the result of the HbA1c measurement and could therefore be taken into account, if desired, so as not to be a source of error in the HbA1c measurement.

In an embodiment of the present invention, the method comprises the possible following steps:

a) separation of blood cells from plasma in the blood sample (S1);

b) lysis of blood cells in order to release the hemoglobin molecules (S2);

c) addition of aggregated silver colloid to the hemoglobin molecules (S3);

d) Surface Enhanced Raman Scattering measurement of the hemoglobin molecules adsorbed onto the silver colloid (S4);

e) analysis of the measured data in order to obtain a Raman spectrum (S5);

f) deduction of the glycated hemoglobin (HbA1c) concentration from the spectrum (S6).

In another embodiment of the present invention, the method comprises, between step a) and step b), a further step consisting in incubation of the cell fraction obtained in step a) with a saline solution to remove labile glycated hemoglobin with a Schiff base (pre-HbA1c).

The invention also relates to a device for quantitative measurement of glycated hemoglobin (HbA1c) in a blood sample containing hemoglobin molecules, comprising:
means for extracting the hemoglobin molecules from the blood sample,
means for adsorbing the hemoglobin molecules onto a roughened metal surface and
means for analyzing the spectrum obtained by a Surface Enhanced Raman Scattering of the adsorbed molecules.

According to an embodiment, the means for extracting the hemoglobin molecules from the blood sample comprise a first module for the separation of blood cells from plasma and a second module for the extraction of glycated and normal hemoglobin molecules from the blood cells.

According to an embodiment, the means for adsorbing the hemoglobin molecules onto a roughened metal surface comprise a first source and means for contacting the extracted molecules with an aggregating agent and a second source and means for contacting the extracted molecules with silver colloidal particles.

According to an embodiment, the means for analyzing the spectrum obtained by a Surface Enhanced Raman Scattering of the adsorbed molecules comprise a measurement chamber, a light source and a detection unit for the detection of Surface Enhanced Raman Scattering signals.

According to an embodiment, the device is automated.

These and other aspects of the invention will be more apparent from the following description with reference to the attached drawings.

### BRIEF DESCRIPTION OF THE DRAWINGS

FIG. 1 is a schematic block diagram representing an example of a device for implementing an embodiment of the method of the present invention;

FIG. 2 illustrates graphically in a schematic way the SERRS spectrum of normal hemoglobin (in dotted line) and glycated hemoglobin (in full line);

FIG. 3 is a block diagram representing the main steps of an embodiment of the method of the present invention and

FIG. 4 is a schematic block diagram representing an example of an integrated device for automated quantitative measurement of glycated hemoglobin (HbA1c) according to an embodiment of the invention.

### DETAILED DESCRIPTION OF THE EMBODIMENTS

The invention seeks to measure the concentration of HbA1c by Surface Enhanced Raman Scattering (SERS). Hemoglobin molecules are extracted from the blood sample and adsorbed to a roughened metal surface, and then submitted to the beam of a light source, such as a laser, in order to obtain SERS signals.

Raman spectroscopy is based on the following phenomenon: when a compound, called the analyte, is illuminated with an appropriate light source, the vast majority of reflected photons are emitted with an energy (frequency) identical to that of the incident light (Rayleigh scattering), while a small number of photons emerge with altered energy levels resulting from the phenomenon known as “Raman scattering”, due to vibrations of molecules. This scattering (also referred to as backscattering) of light by the molecules of the sample is detected and a molecular specific vibrational spectrum is obtained. Each peak of the spectrum corresponds to a particular bound for a component. As a consequence, the resulting Raman spectrum is characteristic of the chemical composition and structure of the light absorbing molecules in the sample, while the intensity of the Raman scattering is depending on the concentration of these molecules. Raman spectroscopy is a reliable technique, since different compounds display different Raman responses, each spectrum being therefore unique for a particular analyte.

The basic approach to obtain SERS signals is to have the target analyte adsorbed onto a suitable roughened metal surface. The expression “roughened metal surface” herein designates either a suspension of colloidal metal nano-particles or a solid metal substrate; the metal could be, for instance, gold, silver or copper, preferably silver, since the Raman excitation of hemoglobin is performed, in one embodiment of the invention, by means of light with a wavelength between 400 and 600 nm, as will be seen below. According to an embodiment of the invention, silver nano-particles are used, prepared as a colloidal suspension; in order to obtain maximum enhancement, the colloidal particles should be aggregated into discrete clusters, as will be detailed later.

The surface with the target analyte is then irradiated with a laser beam and the scattering collected with a Raman spectrometer. Due to interaction between the adsorbed molecules and the roughened metal surface, the Raman scattered signal is enhanced by several orders of magnitude compared to standard or Resonance Raman scattering.

The signal can even be further enhanced by using a light source excitation wavelength in resonance with (that is to say, close to) a maximum absorption frequency of the target analyte. This corresponds to Resonant Raman Scattering (RRS), performed simultaneously with SERS: such a technique is called Surface Enhanced Resonance Raman Scattering (SERRS). With SERRS Raman signal can be enhanced by up to 10¹⁴ times. In SERRS, the light source used to generate the Raman spectrum should be a coherent light source, such as a laser, tuned substantially in the absorption band of the analyte; it could be close to the analyte's absorption maximum wavelength or to a secondary peak in the analyte's absorption spectrum.

Hereinafter, when reference will be made to SE(R)RS, one should understand either SERS or SERRS.

In comparison with other optical detection methods, SE(R)RS presents the advantage that the scattered light consists of molecule-specific vibrational peaks and is very much enhanced by the surface enhancement effect, while it is, as explained above, unique for any Raman active compound (i.e. any molecule where the polarizability can change). The method is highly sensitive, allowing the detection of analytes even at concentrations in the picomolar range.

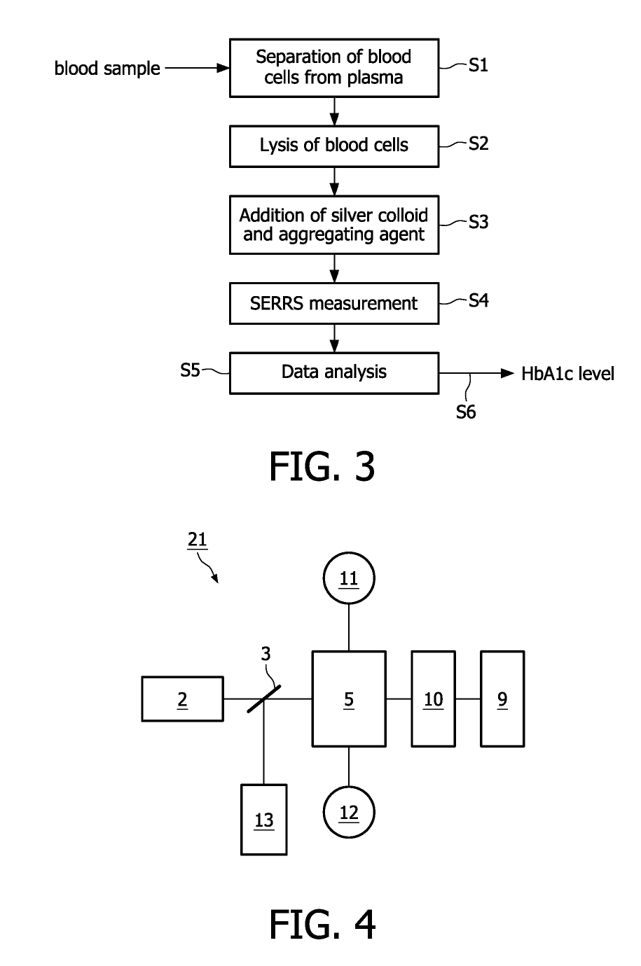

An embodiment of a method for quantitative measurement of glycated hemoglobin (HbA1c) in a blood sample of the present invention will now be described in greater details, with reference to FIG. 3.

In a first step (S1), the red blood cells are isolated from the blood sample, by separating the red blood cells from plasma, for instance here by retaining the cells with a suitable membrane. Any membrane suitable for such a separation may be employed, such as for instance a BTS-SP membrane (polysulfone asymmetric membrane) from Pall Corporation®, or a PlasmaSep® polycarbonate track etch membrane from Osmonics®.

In a second step (S2), a lysis of the blood cells is performed, in order to obtain release of the hemoglobin molecules (glycated and non-glycated) contained in the cells. In an embodiment of the invention, the lysis is obtained by incubation of the blood cells in water or a hypotonic buffer, which makes the cells swell and finally burst due to water uptake, their content being expulsed out of the cell membrane into the solution.

In a third step (S3), silver colloid is added to the solution containing the HbA and HbA1c molecules. in an embodiment of the invention, the silver colloid is citrate-reduced silver colloid (prepared as described by Munro et al. (1995), Langmuir, 11:3712-3720). Typically, the silver colloid consists of nano-particles with a diameter of 40-60 nm.

The silver colloid is aggregated by an aggregating agent, in order to form clusters which give a significantly higher enhancement of the Raman scattering than single nano-particles. The aggregating agent can for instance be an inorganic salt such as sodium chloride or sodium nitrate, or an amine such as spermine. In fact, the citrate-reduced silver colloid can bind all the particles with a suitable surface charge which are present in the solution and, especially, HbA1c molecules as well as HbA molecules.

In a fourth step (S4), the solution containing the aggregated colloid of silver nano-particles and the hemoglobin molecules (of the HbA type and of the HbA1c type), is analyzed by a SE(R)RS measurement.

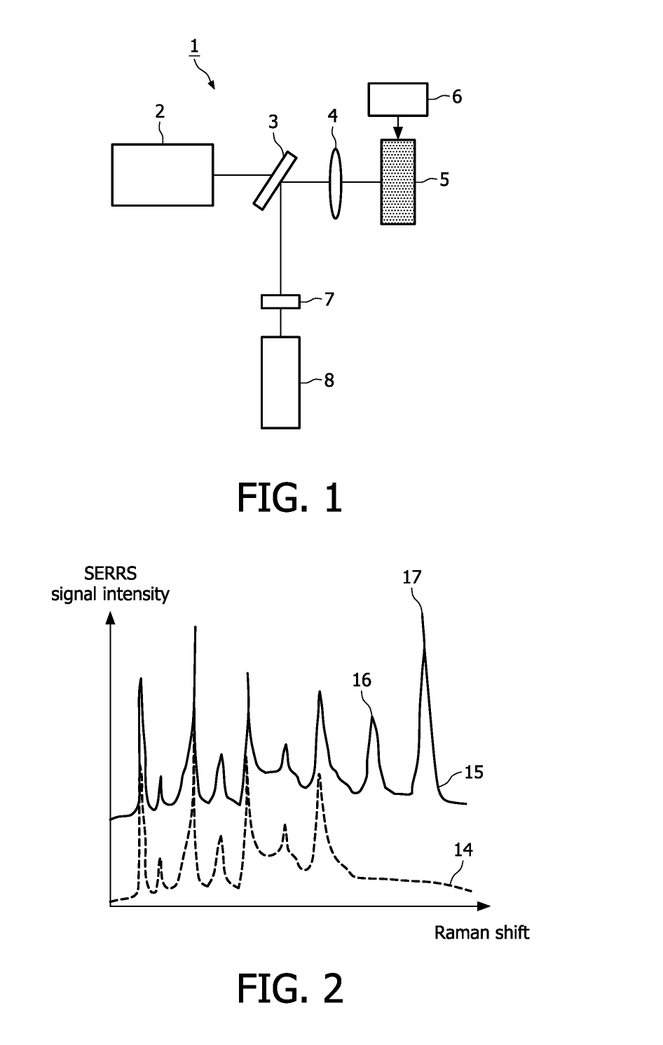

A device 1 for measurement of HbA1c concentration by SE(R)RS according to an embodiment of the invention will now be described, with reference to FIG. 1. The device 1 comprises a light source 2, here a laser 2, a beam splitter 3, a lens 4, a measurement chamber 5, a module 6 for sample preparation (in which the previously described steps S1 to S3 are performed), a filter 7 and a Raman spectrometer 8. Any standard Raman spectrometer can be used to obtain SE(R)RS.

The laser 2 emits an excitation beam, which can possibly be spectrally purified with a bandpass filter. The laser beam is directed towards the sample preparation through the beam splitter 3 and the focusing lens 4. The scattering light is gathered in this same lens 4 and directed by the beam splitter 3, which imposes a right-angle path to the light. The light passes through the filter 7, where the Rayleigh scattering light is removed from the light beam, so that the beam contains the Raman scattering light only and therefore the Raman signals. The Raman signals are detected by the Raman spectrometer 8. The Raman spectrometer 8 contains a grating to spatially resolve or disperse the Raman scattered light into its spectral components onto the detector. The detector can be of any type from the prior art; it can for instance be a charge coupled device detector (CCD).

An integrated device 21 for automated quantitative measurement of HbA1c concentration by SE(R)RS according to a particular embodiment of the invention will now be described, with reference to FIG. 4. The elements of the device 21 which are similar to the ones of the device 1 of FIG. 1 are designated with the same numeral references.

The device 21 comprises a first module 9 for the separation of blood cells from plasma, a second module 10 for the extraction of glycated and normal hemoglobin molecules from the blood cells, a first source and means 11 for contacting the extracted molecules with an aggregating agent, a second source and means 12 for contacting the extracted molecules with silver colloidal particles, a measurement chamber 5, a light source 2 and a detection unit 13 for the detection of SE(R)RS signals.

More precisely, the blood sample to be analyzed is inserted into the first module 9 where the blood cells are separated from the blood plasma, that is to say, where the first step (S1) described above is implemented. This separation may be achieved by the use of a suitable membrane such as a BTS-SP membrane (polysulfone asymmetric membrane) from Pall Corporation®, or a PlasmaSep® polycarbonate track etch membrane from Osmonics®. In an alternative embodiment, centrifugational forces are used to sediment the blood cells.

Subsequently the blood cells are transferred to the second module 10 which contains a suitable buffer for the extraction of hemoglobin molecules by lysis of the blood cells; module 10 is therefore arranged for implementing the second step (S2) described above. In an embodiment, lysis of the cells to release both normal and glycated hemoglobin molecules may be accomplished by incubation with a hypotonic buffer such as 10 mM Tris [pH 7.6], 5 mM MgCl2, 10 mM NaCl. Alternatively, commercially available lysis buffers may be used, e.g. RBC lysis buffer (eBiosciences®), or erythrocyte lysis buffer (Qiagen®).

The extracted hemoglobin molecules are transferred to the measurement chamber 5 where an aggregating agent (e.g. spermine) and silver colloidal particles are added from sources 11 and 12, that is to say, the third step (S3) described above is implemented.

After adsorption of the hemoglobin molecules onto the surface of the aggregated silver colloid, the solution is excited with laser light from the light source 2 and SE(R)RS signals are detected by the detection unit 13. The detection unit 13 typically comprises a filter and a Raman spectrometer, as in the device 1 of FIG. 1.

The modules 9, 10 and the sources 11, 12 constitute module 6 for sample preparation. This module 6 may comprise other elements. The module 6 for sample preparation of FIG. 1, which has not been described in details, may comprise similar modules and sources; it may otherwise comprise different suitable elements, since the device 1 of FIG. 1 is operated manually. The main difference between the device 1 of FIG. 1 and the device 21 of FIG. 4 is that the device 21 of FIG. 4 is automated. The whole method implemented on the device 21 of FIG. 4 may be monitored by a computer.

Even if not shown on FIG. 4, the device 21 may also comprise a lens similar to the lens 4 of FIG. 1.

The device 21 may also comprise a means for data analysis such as a computer with suitable software that calculates the amount of glycated hemoglobin from the measured SE(R)RS signals.

The integrated device 21 allows to carry out the method of the invention in an automated way.

Other devices may be used to implement the method of the invention.

Whatever the device is, it may be noted now that, if the method is performed with the goal to obtain SERS signals, the wavelength of the laser 2 depends preferably on the laser used than on the analyte (e.g. 514 nm). If the method is performed with the goal to obtain SERRS signals, the wavelength of the laser 2 is preferably in an absorption band of the analyte, as explained above. That is, the laser wavelength is close to or around one of the maxima of the HbA1c absorption spectrum. Since the absorption spectrum of HbA1c is about the same as the one of HbA, it is possible to use a wavelength of the absorption spectrum of HbA, which is well known, in order to obtain enhancement for HbA1c.

HbA presents absorption bands around 412 nm, 543 nm and another around 577 nm. The preferred practical excitation wavelengths for SE(R)RS on Ag colloids lie more between 520 and 600 nm, e.g. at or around 543 nm or 577 nm. For other metals the preferred wavelengths may be different, for instance for gold with Plasmon resonances more in the red wavelength range it would be more practical to use wavelengths between 550 and 650 nm (e.g. 632.8 nm (HeNe laser)).

The excitation with the laser 2 stimulates in any case a SE(R)RS signal from both the glycated and the normal hemoglobin molecules contained in the measurement chamber 5. According to a particular embodiment of the present invention, the concentration of HbA is measured simultaneously with the concentration of HbA1c, on the basis of the same SE(R)RS spectrum. In such a case, should the method of measurement be SERRS, the laser wavelength is beneficially chosen close to or around a maximum absorption wavelength of HbA, since Raman signals from the HbA molecules as well as from the HbA1c molecules will be resonance enhanced by the use of an excitation frequency near a HbA maximum absorption wavelength.

In a fifth step (S5), the measured data are analyzed. The apparatus for obtaining and/or analyzing the SE(R)RS spectrum may include some form of data processor such as a computer. Once the SE(R)RS signal has been captured by the detector 8, its frequency and intensity data are passed over to this computer for analysis.

The SE(R)RS spectrum obtained may for instance be of the type of the spectrum of FIG. 2. This spectrum is represented on a graph with the Raman shift in abscissa and the SE(R)RS signal intensity in ordinate; the graph of FIG. 2 represents a SERRS signal.

The spectrum in dotted line 14 corresponds to normal hemoglobin HbA, while the spectrum in full line 15 corresponds to glycated hemoglobin HbA1c. Due to the structural difference the signal obtained from HbA1c contains some additional spectral features in comparison with the signal due to HbA, which can be observed on FIG. 2 as two additional peaks 16, 17; this permits to discriminate the two spectra. Each bound between glucose and hemoglobin gives different peaks, but one peak different from the HbA spectrum is in fact sufficient to discriminate the spectra.

In a sixth step (S6), the HbA1c concentration (or level) is deduced from an analysis of the measured spectrum; more precisely, this concentration is deduced from the SE(R)RS signal intensity. In the particular embodiment described above, wherein HbA concentration is also measured, a multivariate method is implemented to determine the concentration of both glycated and normal hemoglobin in the sample.

According to another embodiment of the invention, the method contains a further step, which is comprised between the first step (S1) and the second step (S2)—that is to say, before the lysis of the red blood cells—which consists in incubation of the cell fraction (obtained by passing the blood through a membrane) with a saline solution to remove labile glycated hemoglobin with a Schiff base, namely pre-HbA1c. This step is optional. If this step is not performed, then the SE(R)RS signal will incorporate the Raman scattering of the pre-HbA1c molecules and this should be taken into account in the final result of the HbA1c concentration; however, taking this into account is not that complicated, since in SE(R)RS pre-HbA1c can easily be distinguished due to the Schiff base which gives distinct spectral features. If this step is performed, then the Raman scattering is not influenced by pre-HbA1c molecules since they have been removed.

Normal ranges for HbA1c usually lie between about 4 to 6% of the total hemoglobin concentration. The concentration for well-controlled diabetic people may be so high as 7 or 8%, and can reach 15 to 20% in case of uncontrolled diabetes. It rarely exceeds 20%. Each 1% change in HbA1c concentration represents an approximate 25 to 35 mg/dL change in average blood glucose, thereby giving a measure for the long-term glycemic status.

In addition, the measurement also yields information on the amount of normal hemoglobin, which can be measured if desired, as explained above in relation to a particular embodiment of the invention. Furthermore, the measurement identifies other variations in the hemoglobin spectrum which may indicate hemoglobin abnormalities; those abnormalities could be a source of error in the prior art methods for determination of HbA1c, while they can be taken into account in the method of the present invention.

In one embodiment of the invention, before implementing the method of the invention, tests are made on samples with a known concentration of HbA1c, which permits to spot the emission peaks of HbA1c and to have a reference to which the subsequent measurements will be compared.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1- Method for quantitative measurement of glycated hemoglobin (HbA1c) in a blood sample containing hemoglobin molecules, the method comprising:
extracting the hemoglobin molecules from the blood sample,
adsorbing the hemoglobin molecules onto a roughened metal surface and
analyzing the spectrum obtained by a Surface Enhanced Raman Scattering of the adsorbed molecules for the quantitative measurement of glycated hemoglobin in the blood sample.

2- Method according to claim 1, wherein the Raman scattering is further enhanced by means of Resonance Raman Scattering.

3- Method according to claim 2, wherein the Raman scattering measurement being performed with a laser (2), the laser wavelength is chosen in a normal hemoglobin (HbA) absorption band.

4- Method according to claim 1, wherein the concentration of normal hemoglobin (HbA) and the concentration of glycated hemoglobin (HbA1c) are determined simultaneously.

5- Method according to claim 1, which comprises the following steps:
a) separation of blood cells from plasma in the blood sample (S1);
b) lysis of blood cells in order to release the hemoglobin molecules (S2);
c) addition of aggregated silver colloid to the hemoglobin molecules (S3);
d) Surface Enhanced Raman Scattering measurement of the hemoglobin molecules (S4);
e) analysis of the measured data in order to obtain a Raman spectrum (S5);
f) deduction of the glycated hemoglobin (HbA1c) concentration from the spectrum (S6).

6- Method according to claim 5, comprising, between step a) and step b), a further step consisting in incubation of the cell fraction obtained in step a) with a saline solution to remove labile glycated hemoglobin with a Schiff base (pre-HbA1c).

7- Device (1, 21) for quantitative measurement of glycated hemoglobin (HbA1c) in a blood sample containing hemoglobin molecules, comprising:
means (9, 10) for extracting the hemoglobin molecules from the blood sample,
means (11, 12) for adsorbing the hemoglobin molecules onto a roughened metal surface and
means ((2, 5, 7, 8), (2, 5, 13)) for analyzing the spectrum obtained by a Surface Enhanced Raman Scattering of the adsorbed molecules.

8- Device according to claim 7, wherein the means (9, 10) for extracting the hemoglobin molecules from the blood sample comprise a first module (9) for the separation of blood cells from plasma and a second module (10) for the extraction of glycated and normal hemoglobin molecules from the blood cells.

9- Device according to claim 7, wherein the means (11, 12) for adsorbing the hemoglobin molecules onto a roughened metal surface comprise a first source and means (11) for contacting the extracted molecules with an aggregating agent and a second source and means (12) for contacting the extracted molecules with silver colloidal particles.

10- Device according to claim 7, wherein the means ((2, 5, 7, 8), (2, 5, 13)) for analyzing the spectrum obtained by a Surface Enhanced Raman Scattering of the adsorbed molecules comprise a measurement chamber (5), a light source (2) and a detection unit ((7, 8), 13) for the detection of Surface Enhanced Raman Scattering signals.

11- Device according to claim 7, which is automated.
